(19) United States
(12) Patent Application Publication
(10) Pub. No.: US 2022/0301711 A1
(43) Pub. Date: Sep. 22, 2022

(54) Systems and Methods for Machine Learning-based Identification of Acute Kidney Injury in Trauma Surgery and Burned Patients

(71) Applicant: The Regents of the University of California, Oakland, CA (US)

(72) Inventors: Hooman H. RASHIDI, Sacramento, CA (US); Nam K TRAN, Sacramento, CA (US)

(21) Appl. No.: 17/617,883

(22) PCT Filed: Jun. 4, 2020
(86) PCT No.: PCT/US20/36170

§ 371 Date: Dec. 9, 2021

Related U.S. Application Data
(60) Provisional application No. 62/860,228, filed on Jun. 11, 2019.

(51) Int. Cl. G16H 50/20; G01N 33/68; G16H 50/30; G16H 10/40

(52) U.S. Cl. CPC G16H 50/20; G01N 33/6893; G16H 50/30; G16H 10/40; G01N 2800/347

(57) ABSTRACT

In some aspects, the disclosure is directed to methods and systems for machine learning-based identification of acute kidney injury in trauma surgery and burned patients. A set of biomarker and vital sign measurements of a population with a known clinical diagnosis may be collected and normalized. A first subset of the modified set of biomarker and vital sign measurements may be used to train a neural network, and a second subset of the modified set of biomarker and vital sign measurements may be used for validation.

### RELATED APPLICATIONS

This application is a national stage entry of Patent Cooperation Treaty Application No. PCT/US2020/036170, entitled “Systems and Methods for Machine Learning-based Identification of Acute Kidney Injury in Trauma Surgery and Burned Patients,” filed Jun. 4, 2020; which claims the benefit of and priority to U.S. Provisional Patent Application No. 62/860,228, entitled “Systems and Methods for Machine Learning-based Identification of Acute Kidney Injury in Trauma Surgery and Burned Patients,” filed Jun. 11, 2019, the entirety of each of which is incorporated by reference herein.

### FIELD OF THE DISCLOSURE

This disclosure generally relates to systems and methods for machine learning and artificial intelligence. In particular, this disclosure relates to systems and methods for machine learning-based identification of acute kidney injury in trauma surgery and burned patients.

### BACKGROUND OF THE DISCLOSURE

Acute kidney injury (AKI) is a common complication among critically ill patients. Severely burned patients, in particular, have been shown to be at high-risk with up to 58% experiencing AKI. The early recognition of AKI helps guide fluid resuscitation and titrate dosing of nephrotoxic drugs in these populations. Unfortunately, traditional biomarkers of renal function such as creatinine and urine output (UOP) have been shown to be inadequate at predicting AKI. Novel AKI biomarkers have been proposed, but widespread use in the United States remains limited. Even with such novel biomarkers, implementations may be slow or inefficient, resulting in delays in treatment, and may tie up physician resources.

### BRIEF DESCRIPTION OF THE DRAWINGS

The patent or application file contains at least one drawing executed in color. Copies of this patent or patent application publication with color drawings will be provided by the Office upon request and payment of the necessary fee.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is an illustration of various implementations of artificial intelligence/machine learning approaches;

FIG. 2 is an illustration of bar graphs of accuracy for different artificial intelligence/machine learning techniques, according to some implementations;

FIGS. 3A and 3B are graphs illustrating comparisons of receiver operator characteristic curves and average areas under the curve for each of a plurality of artificial intelligence/machine learning techniques, according to some implementations;

FIG. 4 is an illustration of an improved workflow for AKI prediction, according to some implementations;

FIG. 5 is a block diagram of an implementation of a machine learning-based system for AKI prediction;

FIG. 6 illustrates graphs showing comparisons of receiver operator characteristic curves and average areas under the curve for proof of concept testing of various biomarkers, according to some implementations;

FIG. 7 is an example heat map illustrating correlation of features to AKI or no-AKI used in building a machine learning model, according to some implementations;

FIG. 8 illustrates graphs showing accuracy of machine learning predictions for an example proof of concept test, according to some implementations;

FIG. 9A is an illustration of an improved workflow for AKI prediction using machine learning, according to some implementations;

FIG. 9B is a flow chart of an implementation of a method for machine-learning based diagnosis and treatment; and

FIGS. 10A and 10B are block diagrams depicting embodiments of computing devices useful in connection with the methods and systems described herein.

The details of various embodiments of the methods and systems are set forth in the accompanying drawings and the description below.

### DETAILED DESCRIPTION

For purposes of reading the description of the various embodiments below, the following descriptions of the sections of the specification and their respective contents may be helpful:
Section A describes embodiments of systems and methods for early recognition of acute kidney injury in trauma surgery and burned patients via artificial intelligence and machine learning techniques;
Section B describes example and proof of concept implementations of systems and methods for artificial intelligence and machine learning for predicting acute kidney injury in severely burned patients; and
Section C describes a computing environment which may be useful for practicing embodiments described herein.

### A. Systems and Methods for Early Recognition of Acute Kidney Injury in Trauma Surgery and Burned Patients via Artificial Intelligence and Machine Learning Techniques

Acute kidney injury (AKI) is a common complication among critically ill patients. Severely burned patients, in particular, have been shown to be at high-risk with up to 58% experiencing AKI. The early recognition of AKI helps guide fluid resuscitation and titrate dosing of nephrotoxic drugs in these populations. Unfortunately, traditional biomarkers of renal function such as creatinine and urine output (UOP) have been shown to be inadequate at predicting AKI. Novel AKI biomarkers have been proposed, but widespread use in the United States remains limited. Even with such novel biomarkers, implementations may be slow or inefficient, resulting in delays in treatment, and may tie up physician resources.

Kidney injury doubles burn mortality—thus, early prediction of acute kidney injury (AKI) in the burn population could benefit from artificial intelligence (AI) and machine learning (ML). This disclosure discusses performances of such AI/ML algorithms and describes generalizable models that may be implemented to augment AKI recognition.

Advances in computational technology and artificial intelligence (AI) and machine learning (ML) may aid in the diagnosis of several disease and may augment the performance of existing tests success. AI/ML using a k-nearest neighbor (k-NN) approach may augment the identification of AKI in burn patients using only plasma creatinine, UOP and N-terminal pro-B-type natriuretic peptide (NT-proBNP). These algorithms may apply to burn victims, as well as other critically ill populations.

Severely burned patients have been shown to be fundamentally different from traditional trauma populations. However, AKI classification remains the same between both populations and based on the Kidney Disease and Improving Global Outcomes (KDIGO) criteria. Notably, many implementations of the KDIGO criteria rely solely on UOP and creatinine measurements, and have poor performance in burn patients. By using the techniques described herein, AI developed in burn patients may be translated to non-burned trauma patients to achieve better performance than KDIGO implementations not utilizing ML techniques. The systems and methods discussed herein are directed to a burn-trained AL algorithm generalized to a non-burned population. This disclosure also provides an evaluation of the performance of KDIGO against ML.

Various implementations of AI/ML algorithms for early recognition of AKI in a combined population of burn and non-burned trauma surgery patients are discussed and compared below, and in particular, AI/ML prediction within the first 24 hours due to burn- and/or trauma injury-related shock (being common mechanisms causing AKI). These algorithms were first trained and validated on a retrospective burn AKI dataset, and then analyzed for generalizability in a second dataset containing a mix of burned and non-burned trauma surgery patients.

Specifically, two databases containing patients (Cohort A and Cohort B) that received neutrophil gelatinase associated lipocalin (NGAL), creatinine, N-terminal pro-B-type natriuretic peptide (NT-proBNP) and urine output (UOP) measurements at admission were used to train, test, and generalize the AI/ML models. Models were first optimized in Cohort A for predicting AKI in Cohort B. Cohort A (n=50) was based on a retrospective dataset of adult (age ≥18 years) burn patients, while Cohort B (n=51) consisted of prospectively enrolled adult burned or non-burned trauma patients at risk for AKI. A grid search and cross validation approach was employed in building 68,100 unique ML models from five distinct ML approaches: logistic regression (LR), k-nearest neighbor (k-NN), support vector machine (SVM), random forest (RF), and deep neural networks (DNN) which enabled us to find the most accurate ML models.

The best generalization accuracy (86%), sensitivity (91%), and specificity (85%) with NGAL alone was noted with LR, SVM and RF models. Generalizability prediction accuracy, sensitivity and specificity were respectively highest with the optimized DNN model (92%, 100%, and 90%) and the k-NN model (92%, 91%, and 93%) when tested with Cohort B using all four biomarkers. k-NN provided best generalization accuracy (84%) without NGAL using only NT-proBNP and creatinine, followed by DNN using creatinine only with an accuracy of 82%. AI/ML algorithms using results obtained at admission accelerated the average (SD) time to AKI prediction by 61.8 (32.5) hours.

These procedures are described in more detail below.

Retrospective Burn Study Population (Cohort A): The retrospective quality database consisted of 50 adult (age ≥18 years) patients with ≥20% total body surface area (TBSA) burns at risk for AKI reported previously. This database was derived from a hospital clinical laboratory project to validate a commercially available plasma neutrophil gelatinase associated lipocalin (NGAL) enzyme linked immunosorbent assay (Bioporto, Inc, Denmark). NGAL testing was performed on residual plasma chemistry samples collected at the time of burn intensive care unit admission. Briefly, NGAL is a novel AKI biomarker and is released by neutrophils during inflammation and renally cleared. During AKI, decreases in glomerular filtration rate (GFR) increases plasma concentrations of NGAL during AKI. Unique to NGAL, renal tubular cells also produce the biomarker during AKI—increasing both plasma and urine concentrations of NGAL.

In addition to NGAL, natriuretic peptide testing was included, given that AKI can lead to acute heart dysfunction and manifest as cardiorenal syndrome. Specifically, N-terminal pro B-type natriuretic peptide (NT-proBNP) was also measured (Roche Diagnostics, Indianapolis, Ind.) using the same plasma samples. Paired to the NGAL and NT-proBNP results, UOP was recorded, as well as plasma creatinine results, and vital signs from the electronic medical record (EMR). Chart review was used to determine which patients experienced AKI during the first one-week of burn intensive care unit admission based on KDIGO criteria.

Prospective Burn and Trauma Population (Cohort B): The second dataset consisted of 51 adult patients with ≥20% TB SA burns or non-burn trauma-related injuries requiring surgery. Inclusion of a non-burned trauma population served to determine the generalizability of each AI/ML model. These patients were prospectively enrolled to obtain residual clinical plasma samples within the first 24 hours of admission for testing by the same NGAL and NT-proBNP assays to predict AKI. Both NGAL and NT-proBNP results were not used for patient care. Again, chart review was performed to obtain paired UOP and plasma creatinine results, as well as patient history, vital signs (i.e., mean arterial pressure, central venous pressure) and demographic data. KDIGO criteria¹⁷ was used to determine AKI status within the first week of stay.

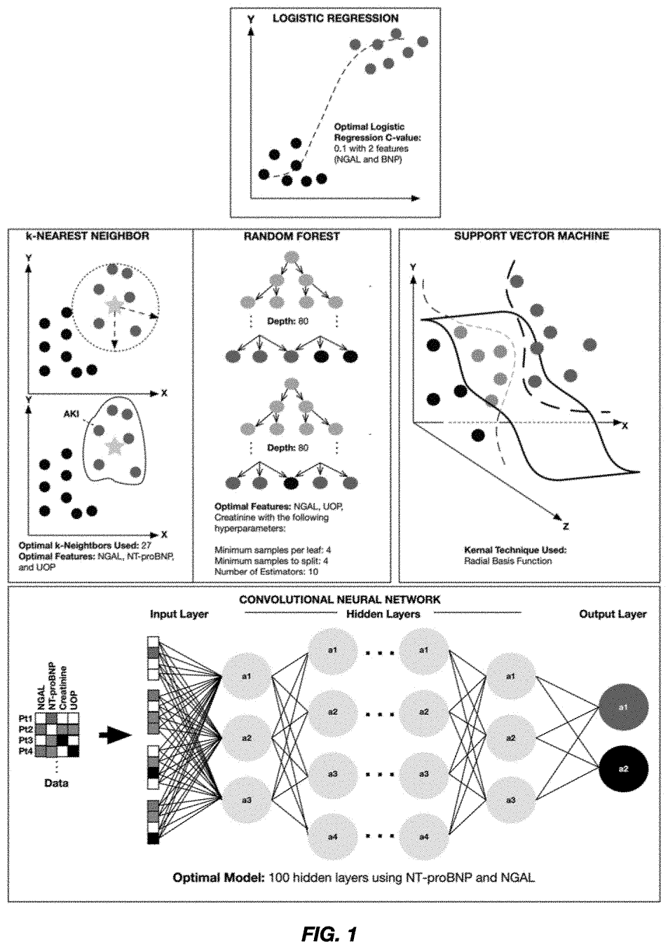

AI/ML Algorithms: Various AI/ML approaches, illustrated in FIG. 1, were evaluated to differentiate AKI versus non-AKI patients. The figure compares the five AI/ML techniques used in the study and illustrated as conceptual drawings. At the top, is LR. Middle row from left to right is k-NN, RF, and SVM respectively. The bottom row illustrates a DNN. Cohort A was used for the initial training and testing. This was then followed by Cohort B serving as means to evaluate the overall generalizability of the ML algorithms. These ML approaches included: (a) logistic regression (LR), (b) k-nearest neighbor (k-NN), (c) random forest (RF), (d) support vector machine (SVM), and a multi-layer perceptron (MLP) deep neural network (DNN), as shown in FIG. 1. Scikit-Learn's version 0.20.2 was used for all five algorithms, though other versions or provider systems may be utilized in different implementations. Briefly, LR is based on traditional statistical techniques identifying predictors of a binary outcome (e.g., AKI vs. no AKI). k-NN is a non-parametric pattern recognition algorithm used for classification and regression. Classification is based on the number of k neighbors and its Euclidean distance (d) from a pre-defined point. In contrast, random forest, a form of ensemble learning, uses a multitude of constructed decision trees for classification and regression. Next, SVM is a form of AI/ML that classifies data by defining a hyperplane that best differentiates two groups (e.g., AKI vs. non-AKI patients) by maximizing the margin (the distance), ultimately leading to a hyperplane-bounded region with the largest possible margin. Thus, the goal of SVM is to maximize the distance (margin) between groups of data which can also be applied as a linear method to nonlinear data by transposing the data features into a higher dimension (e.g., three dimensions) through the use of kernels. This ultimately allows for a better classification and differentiation of the groups of interest (e.g., AKI versus No-AKI). Lastly, DNN utilizes artificial neural networks with multiple levels between input and output layers. Ultimately these multi-layer perceptrons (MLP) within the DNN identifies the appropriate mathematical manipulation to convert an input into an output. For this study, nearly 2,000 unique ML neural network models were generated through a custom multi-layer neural network grid search in Scikit learn library. The “Adam” solver (a stochastic gradient-based optimizer) was used within the custom multi-layer neural networks along with a grid search with variable number of hidden layers, variable penalty regularization alpha parameters, variable tol values (tolerance for the optimization parameters) and two unique activation functions: ReLU (the rectified linear unit function) and tanh (hyperbolic tan function) to find the best performing multi-layer neural network for each category. Since these ML algorithms are sensitive to unscaled data, variables were scaled based on a standard scaler method transforming features to a mean of 0 with a standard deviation of 1. In the example implementation illustrated, each patient (Pt #) data matrix containing various combinations of biomarkers and their respective levels (white: none, grey: low, black: high) are processed by hidden layers for classification as having AKI or no AKI.

Cross Validation studies: Cross validation studies were also performed for LR, RF, k-NN, SVM, and DNN methods using the Scikit-learn cross validation grid search tool. This technique along with the grid search hyperparameter variations (noted above) enabled us to build and compare unique models to yield a total of 68,100 ML models. Using this approach, we were able to empirically assess and compare the performance of all these models which ultimately lead to identifying the best performing ML models with a unique set of hyperparameters within each ML method. The mean accuracy for each set of these models were then analyzed.

Statistical Analysis: JMP software (SAS Institute, Cary, N.C.) was used for statistical analysis. Describe statistics were calculated for patient demographics. Continuous variables were analyzed using the 2-sample t-test, while discrete variables were compared using the non-parametric Chi-square test. Multivariate logistic regression was used to determine predictors of AKI with age and burn size serving as covariates. Repeated measures analysis of variance was used for time series data. A p-value <0.05 was considered statistically significant with receiver operator characteristic (ROC) analysis also performed to compare AKI biomarker performance.

Patient demographics and biomarker comparisons between study cohorts (A vs. B, AKI vs. non-AKI, and burned vs. non-burned groups) are shown in Table 1. Briefly, 50% of patients (25/50) in Cohort A experienced AKI within the first week of hospital stay as shown previously. Five patients experienced fluid overload manifested as compartment syndrome. Again, Cohort A served as an AI/ML “training” dataset. In contrast, 21.6% (11/51) of Cohort B patients experienced AKI within the same timeframe. Eight patients experienced over-resuscitation presenting with compartment syndrome (n=2), pulmonary edema (n=2), or both compartment syndrome and pulmonary edema (n=4). Leveraging both some population sim-ilarities and differences, Cohort B was used as our secondary testing dataset to assess the generalizability of the models generated from cohort A. The mean (standard deviation [SD]) time for patients to meet KDIGO AKI criteria was 42.7 (23.2) hours for Cohort A and 71.5 (39.5) hours for Cohort B.

TABLE 1
Patient Demographics and Comparison of Biomarker Levels

| | | |
|---|---|---|
| | Burn AKI | Burn Non-AKI |
| COHORT A - TRAINING | (n = 25) | (n = 25) |

| | | | | |
|---|---|---|---|---|
| Mean (SD) Age (years) | 39.1 | (49.2) | 39.7 | (15.5) |

| | | |
|---|---|---|
| Gender (M/F) | 20/5 | 19/6 |

| | | | | |
|---|---|---|---|---|
| Burn Size (% TBSA) | 49.2 | (24.1) | 43.3 | (18.9) |
| Mean (SD) Arterial | 78.9 | (11.5) | 80.1 | (5.2) |
| Pressure (mmHg) | | | | |
| Mean (SD) Central | 13.3 | (3.4) | 12.0 | (7.6) |
| Venous Pressure (mmHg) | | | | |
| Mean (SD) | 1.21 | (0.51) | 0.90 | (0.22) |
| Creatinine (mg/dL) | | | | |
| Mean (SD) NGAL | 185.1 | (86.3) | 110.3 | (48.1) |
| (ng/mL) | | | | |
| Mean (SD) NT-proBNP | 25.7 | (15.4) | 16.0 | (15.3) |
| (pg/mL) | | | | |
| Mean (SD) UOP | 81.5 | (31.6) | 85.7 | (48.9) |
| (mL/hr) | | | | |

| | | | |
|---|---|---|---|
| Mean (SD) Time to AKI | 42.7 | (23.2) | N/A |
| (time from admission to | | | |
| achieving AKI based on | | | |
| KDIGO criteria in hours) | | | |

| | Burn AKI | | Burn Non-AKI | | Trauma AKI | | Trauma Non-AKI | |
|---|---|---|---|---|---|---|---|---|
| COHORT B - TEST | (n = 6) | | (n = 15) | | (n = 7) | | (n = 23) | |
| Mean (SD) | 38.2 | (41.5) | 40.1 | (20.2) | 37.6 | (39.9) | 39.1 | (19.5) |
| Age (years) | | | | | | | | |
| Gender (M/F) | 4/2 | | 12/3 | | 4/3 | | 15/10 | |
| Burn Size (% TBSA) | 41.1 | (14.8) | 40.0 | (20.4) | N/A | | N/A | |
| Mean (SD) Arterial | 82.8 | (15.5) | 79.7 | (18.3) | 70.3 | (20.8) | 75.1 | (20.3) |
| Pressure (mmHg) | | | | | | | | |
| Mean (SD) Central | 12.6 | (4.4) | 12.9 | (5.8) | 10.7 | (6.2) | 12.3 | (6.9) |
| Venous Pressure (mmHg) | | | | | | | | |
| Mean (SD) Creatinine | 2.15 | (1.77) | 0.93 | (0.46) | 2.16 | (1.57) | 0.86 | (0.32) |
| (mg/dL) | | | | | | | | |
| Mean (SD) | 300.4 | (213.5) | 110.0 | (39.7) | 396.7 | (393.7) | 77.4 | (32.1) |
| NGAL (ng/mL) | | | | | | | | |
| Mean (SD) | 144.3 | (23.6) | 57.5 | (16.9) | 137.3 | (62.1) | 93.7 | (10.4) |
| NT-proBNP (pg/mL) | | | | | | | | |
| Mean (SD) | 47.7 | (41.2) | 93.3 | (41.1) | 66.1 | (37.2) | 87.4 | (58.2) |
| UOP (mL/hr) | | | | | | | | |

| | | | | | | |
|---|---|---|---|---|---|---|
| Mean (SD) Time to AKI | 43.9 | (15.3) | N/A | 82.7 | (38.6) | N/A |
| (time from admission to | | | | | | |
| achieving AKI based on | | | | | | |
| KDIGO criteria in hours) | | | | | | |

Abbreviations: F, female; KDIGO, Kidney Disease: Improving Global Outcomes; M, male; mmHg, millimeters mercury; mL, milliliter; ng, nanogram; NGAL, neutrophil gelatinase associated lipocalin; N/A, not applicable; NT-proBNP; N-terminal pro-B-type natriuretic peptide; pg, picogram; SD, standard deviation; TBSA, total body surface area; and UOP, urine output.

Focusing on Cohort B, which was the study AFIVIL “test/generalizability” population, median (IQR) plasma creatinine (1.17 [1.52] vs. 0.83 [0.53], P<0.001) and UOP (66.4 [79.3] vs. 86.5 [53.6] mL/hour, P=0.023) were statistically different between AKI versus non-AKI groups. NT-proBNP was significantly higher in the AKI group (107.0 [53.3] vs. 60.4 [13.2] pg/mL, P=0.016). NGAL served as an independent predictor of AKI (OR 2.7, 95% CI 0.8-4.5, P<0.001) and concentrations were found to be significantly higher among the AKI patients (260.7 [163.8] vs. 89.6 [38.1] ng/mL, P=0.006). However, there were no statistically significant differences between burned vs. non-burned AKI patients for mean plasma creatinine (2.15 [1.77] vs. 2.16 [1.58] mg/dL, P=0.984), UOP (47.8 [41.2] vs. 66.1 [37.2] mL/hour, P=0.422), and mean NT-proBNP (114.3 [23.6] vs. 137.3 [93.7] pg/mL, P=0.551). The average time from admission to meeting KDIGO AKI criteria was significantly different between burned versus non-burned patients respectively (43.9 [15.3] vs. 82.7 [38.6], P=0.029).

Comparing non-AKI patients with versus without burn injury, mean NGAL levels were significantly higher among the non-burned population (109.9 [39.7] vs. 77.4 [32.1] ng/mL, P=0.013), while mean NGAL levels between burned versus non-burned AKI patients were similar (300.4 [213.5] vs. 396.7 [393.7] ng/mL, P=0.589). Sub-group analysis among Cohort A and B burn patients experiencing fluid overload complications (i.e., compartment syndrome and/or pulmonary edema) showed significantly high mean NT-proBNP levels (Cohort A [n=5]: 78.2 [15.8] pg/mL vs. Cohort B [n=8], 372.4 [10.7] pg/mL, P<0.001). Receiver operator characteristics analysis showed NGAL serving as the best AKI biomarker (area under the curve [AUC]: 0.93, P=0.023), followed by NT-proBNP (0.85), plasma creatinine (0.68), and UOP (0.57). The area under the ROC curve for each biomarker was significantly (P=0.038) larger among non-burned patients versus burned patients.

AI/ML Modeling and Comparisons with Cohort B: Table 2 summarizes the mean accuracy for the AI/ML models during the initial validation phase using Cohort A.

TABLE 2
Mean Accuracy Using Train/Test Dataset (Cohort A)

| | Mean | | | | | | | | | |
|---|---|---|---|---|---|---|---|---|---|---|
| | (SD) Accuracy | | | | | | | | | |
| | (%) | | | | | | | | | |
| Biomarker Combination | DNN | | LR | | k-NN | | SVM | | RF | |
| NGAL, NT-proBNP, UOP, Creatinine | 100 | (0) | 95 | (10) | 95 | (10) | 98 | (8) | 90 | (17) |
| NGAL, UOP, NT-proBNP | 88 | (17) | 88 | (17) | 90 | (17) | 83 | (23) | 90 | (12) |
| NGAL, UOP, Creatinine | 100 | (0) | 98 | (8) | 98 | (8) | 98 | (8) | 93 | (16) |
| NGAL, NT-proBNP, Creatinine | 98 | (8) | 95 | (10) | 95 | (10) | 95 | (10) | 93 | (11) |
| NT-proBNP, Creatinine, UOP | 90 | (17) | 88 | (17) | 93 | (16) | 93 | (16) | 93 | (11) |
| NGAL, NT-proBNP | 93 | (11) | 93 | (11) | 93 | (11) | 90 | (17) | 90 | (17) |
| NGAL, Creatinine | 95 | (10) | 95 | (10) | 95 | (10) | 95 | (10) | 93 | (16) |
| NGAL, UOP | 90 | (17) | 83 | (22) | 90 | (17) | 88 | (17) | 90 | (17) |
| NT-proBNP, Creatinine | 90 | (12) | 88 | (13) | 88 | (13) | 90 | (12) | 90 | (12) |
| NT-proBNP, UOP | 85 | (20) | 85 | (20) | 78 | (21) | 85 | (20) | 90 | (12) |
| Creatinine, UOP | 65 | (20) | 48 | (18) | 65 | (20) | 60 | (20) | 60 | (23) |
| NGAL | 85 | (17) | 83 | (16) | 85 | (17) | 85 | (17) | 85 | (17) |
| Creatinine | 68 | (16) | 58 | (39) | 65 | (32) | 68 | (20) | 65 | (20) |
| UOP | 58 | (16) | 30 | (19 | 48 | (13) | 43 | (20) | 50 | (25) |

Abbreviations: DNN, deep neural network; k-NN, k-nearest neighbor; LR, logistic regression; NGAL, neutrophil gelatinase associated lipocalin; NT-proBNP; N-terminal pro-B-type-natriuretic peptide; RF, random forest; SVM, support vector machine; and UOP, urine output

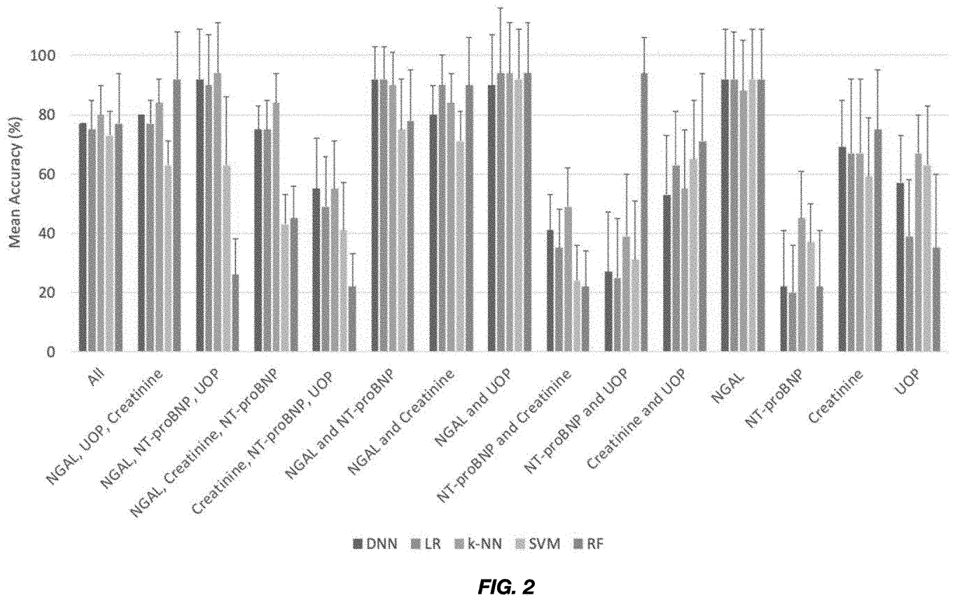

FIG. 2 is an illustration of bar graphs showing bar graphs accuracy for each of the five AI/ML techniques with differing combinations of NGAL, UOP, plasma creatinine, and NT-proBNP. Standard deviations are shown as error bars. Data was based on Cohort B (n=51) severely burned or non-burned trauma patients. For the generalization phase (Cohort B), models using NGAL and NT-proBNP only reported the highest accuracy of 92% and AUC of 0.92 using either DNN or LR. The generalization accuracy and AUC of our NGAL and creatinine only model (90% and 91%) was noted within our LR model. Excluding NGAL and retaining the other biomarkers markedly reduced the predictive performance in all 5 of our ML platforms, DNN, LR, k-NN, SVM and RF (generalization accuracy of 55%, 49%, 55%, 41%, 22% and AUC of 71%, 68%, 68%, 63%, 50%, respectively). Notably, in the absence of NGAL, the highest generalization prediction accuracy and AUC was noted within our RF model using creatinine and UOP only (71% and 75%, respectively) and within our DNN model using the combination of creatinine, UOP, and NT-proBNP (55% and 71%, respectively).

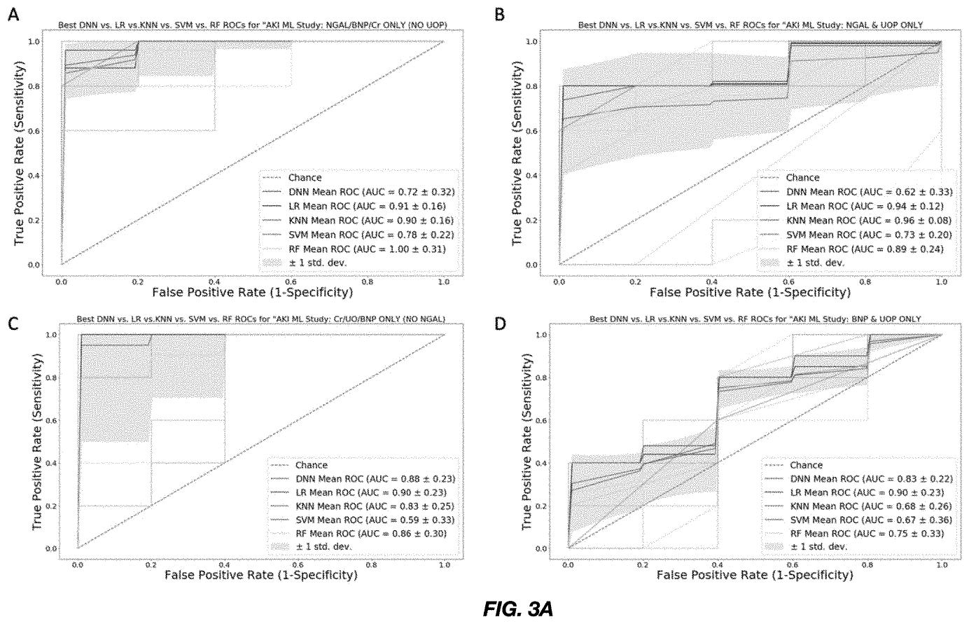

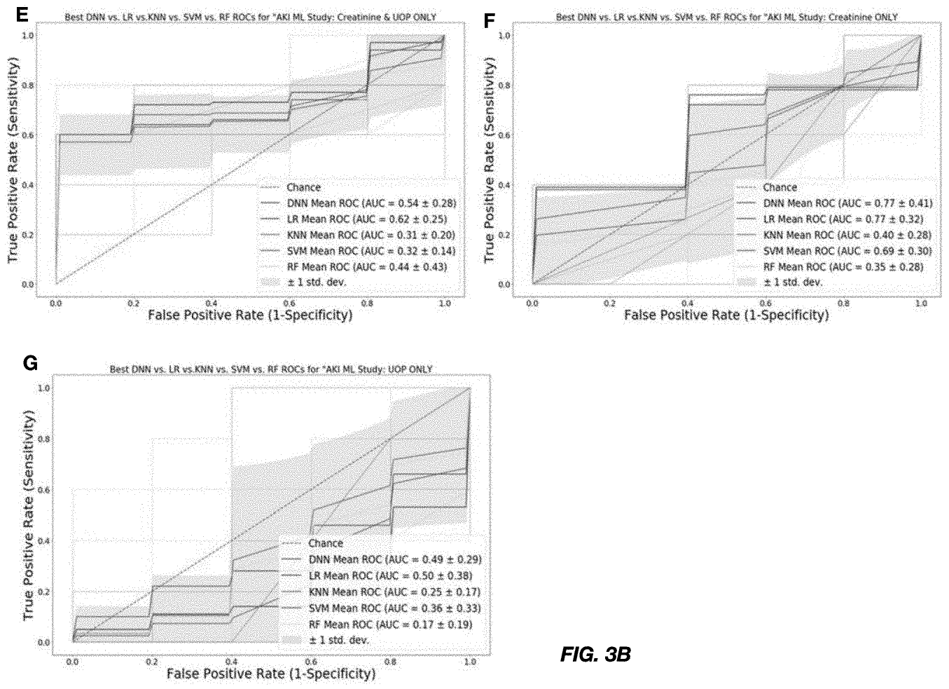

FIGS. 3A and 3B are illustrations comparing ROC curves and average AUCs for each AI/ML model with different combinations of biomarkers tested within Cohort A. These figures compare the best ROC curves for each AI/ML technique with differing combinations of biomarkers. False positive rate (1—specificity) and true positive rates (sensitivity) are reported on the x- and y-axis respectively. Panel A is for NGAL, NT-proBNP, plasma creatinine only. Panel B is for NGAL and UOP only. Panel C is for plasma creatinine, UOP, and NT-proBNP only. Panel D is for NT-proBNP, and UOP only. Panel E is for plasma creatinine and UOP only. Panel F is for plasma creatinine only, and Panel G shows UOP only. Area under the ROC curve values are reported in the bottom right of each Panel. Area under the ROC curve when using all biomarkers were 1.00 (0), 0.96 (0.13), 0.97 (0.06), 0.69 (0.29), and 0.97 (0.07) respectively for DNN (labeled as MLP), LR, k-NN, SVM, and RF.

The generalizability of a burn population derived AI/ML algorithm for predicting AKI was evaluated, concluding that machine learning techniques provide unique advantages in the context of AKI including the potential to be highly automated via electronic medical record systems, and enable early classification of subtle changes for predicting AKI. Various AI/ML methods may be implemented to provide optimal accuracy across the burn-trauma population.

Neutrophil gelatinase associated lipocalin (NGAL) is particularly predictive of AKI in both burn and trauma surgery populations. Higher baseline NGAL levels found in burn patients may be due to their underlying systemic inflammatory response to their injury. Unfortunately, the United States Food and Drug Administration has yet to approve an NGAL which limits the utility of this biomarker for AKI applications, forcing healthcare providers to rely on UOP and plasma creatinine. Urine output has been shown previously to perform poorly for AKI especially in burn critical care. The same holds true for plasma creatinine which exhibits high biological variability and less than ideal inter-assay imprecision.

Instead, according to the systems and methods discussed herein, AI/ML models may be used for analysis and prediction of AKI. In many implementations, DNN may provide best generalization accuracy and balance between sensitivity/specificity using NGAL, UOP, creatinine, and NT-proBNP—achieving an average accuracy of 92% and an AUC of 95%. Performance of k-NN using the same biomarker combination also achieved 92% accuracy, but with a lower AUC (92%). NT-proBNP combined with creatinine using k-NN may be used instead of NGAL as a parameter in some implementations to provide high generalization accuracy.

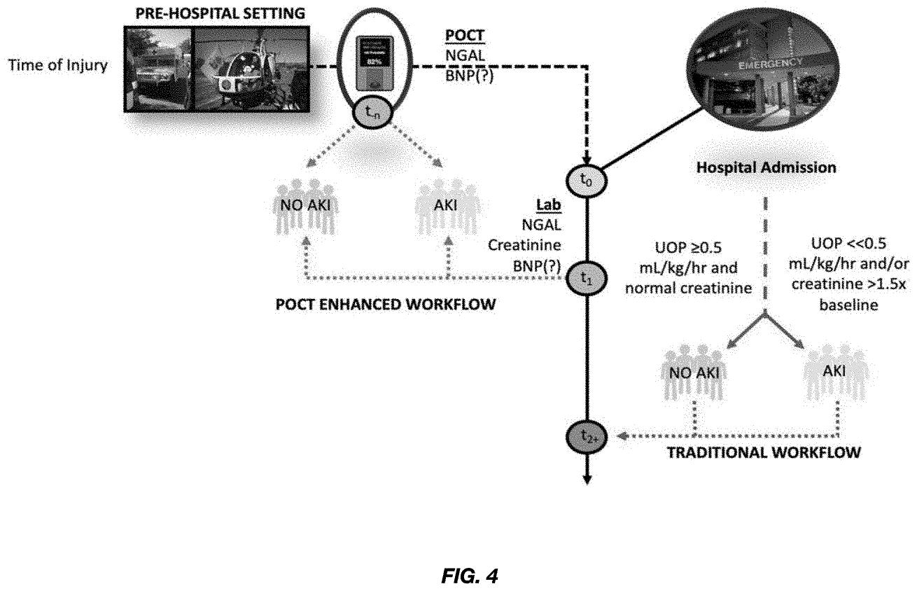

Performance of the k-NN model in Cohort B was similar to burn-focused studies based on Cohort A. In some implementations, AI/ML may augment AKI prediction accuracy with only plasma creatinine and NT-proBNP. These two commonly available biomarkers achieved an accuracy of 84% using the k-NN method. Including UOP with NT-proBNP and plasma creatinine slightly decreased the accuracy for k-NN to 80%, but improved the performance of SVM (80%). One or both of NGAL and NT-proBNP biomarkers may be included in many implementations to increase accuracy. Interestingly, the DNN approach was able to maintain an accuracy of 82% with plasma creatinine only, however the AUC decreased to 0.62. Overall, the AI/ML algorithms predicted AKI an average of 61.8 (32.5) hours before patients met KDIGO criteria. Accordingly, in many implementations, AI/ML may be used in pre-hospital settings (e.g., ambulance, combat casualty evacuations) to augment point-of-care tests which are already available for whole blood creatinine and NT-proBNP testing. FIG. 4 illustrates this temporal improvement graphically.

Combining point-of-care (POC) testing with AI/ML could be used to enhance diagnostic power in pre-hospital settings. The figure illustrates a conceptual diagram where POC creatinine and NT-proBNP testing is used at a pre-hospital admission time (t₋ₙ) point and augmented by AI/ML (green pathways). Point-of-care testing data may be then transmitted to an AI/ML algorithm to predict AKI prior to hospital admission. Alternately, AI/ML may also be employed as early as the first day of admission denoted as t₁. In contrast, traditional workflows (red pathways) relying on urine output and creatinine delay recognition of AKI.

Accordingly, accurate prediction of AKI in a mixed burn/trauma population is feasible using an AI/ML algorithm originally trained for burn patients. This finding highlights the generalizability of AI/ML between these two populations for AKI. Both DNN and k-NN, in particular, provide robust means to predict AKI using both common and esoteric biomarkers of cardiorenal dysfunction. The limited availability and adoption of novel biomarkers such as NGAL increases the appeal of an AI/ML algorithm enhancing the performance of NT-proBNP, UOP, and plasma creatinine for predicting AKI. In particular, NGAL may be analytically superior to traditional AKI biomarkers such as creatinine and UOP in many implementations. With machine learning, the AKI predictive capability of NGAL can be further enhanced and accelerated when combined with NT-proBNP, UOP, and creatinine. Nonetheless, without NGAL, machine learning models continue to provide robust means in accelerating the prediction of AKI using both common and biomarkers of cardiorenal dysfunction.

### B. Example and Proof of Concept Implementations of Artificial Intelligence and Machine Learning for Predicting Acute Kidney Injury in Severely Burned Patients

As discussed above, burn critical care represents a high impact population that may benefit from artificial intelligence and machine learning (ML). Acute kidney injury (AKI) recognition in burn patients could be enhanced by ML. To provide proof of concept, ML models using the k-nearest neighbor (k-NN) algorithm were developed. The ML models were trained-tested with clinical laboratory data for 50 adult burn patients that had neutrophil gelatinase associated lipocalin (NGAL), urine output (UOP), creatinine, and N-terminal B-type natriuretic peptide (NT-proBNP) measured within the first 24 hours of admission.

Half of patients (50%) in the dataset experienced AKI within the first week following admission. ML models containing NGAL, creatinine, UOP, and NT-proBNP achieved 90-100% accuracy for identifying AKI. ML models containing only NT-proBNP and creatinine achieved 80-90% accuracy. Mean time-to-AKI recognition using UOP and/or creatinine alone was achieved within 42.7±23.2 hours post-admission vs. within 18.8±8.1 hours via the ML-algorithm.

Accordingly, in some implementations, the performance of UOP and creatinine for predicting AKI could be enhanced by with a ML algorithm using a k-NN approach when NGAL is not available.

Burn critical care represents a high impact population that may benefit from AI/ML. Early recognition of sepsis and organ dysfunction, both common burn sequelae, could be exploited by AI/ML to integrate various sources of information (e.g., laboratory results, vital signs) into a composite, prognostic, and predictive “clinical picture”. Various platforms (e.g., Scikit-Learn, Apple's Turi Create and Core ML, and Google's Tensor Flow, etc.) may be used to build these very relevant and powerful ML models that could ultimately enhance patient care and health care delivery.

Burn-related acute kidney injury (AKI) is one such focus for AI/ML. Up to 58% of burn patients acquire AKI due to pre-renal (e.g., burn shock, sepsis) and renal mechanisms (e.g., nephrotoxic medications) of injury—with AKI common within the first week due to inadequate resuscitation during the critical first 24 hours of admission. Despite this high prevalence, early recognition remains challenging due to the reliance on serum/plasma creatinine and urine output (UOP) for diagnosing and staging AKI—biomarkers that have known limitations. Creatinine has a slow half-life and exhibits high biological variability. Alternately, UOP may remain unchanged in critically ill patients despite decreasing glomerular filtration rate (GFR). Other novel AKI biomarkers including neutrophil gelatinase associated lipocalin (NGAL), kidney injury marker-1 (KIM-1), tissue inhibitory of metalloprotease-2 (TIMP-2), and insulin-like growth factor binding protein-7 (IGBFP-7) may be utilized to overcome the limitations of UOP and creatinine in some implementations. As discussed herein, in various implementations, ML may be useful in augmenting the predictive power of both traditional and novel indicators of AKI.

To provide proof of concept, a ML study was developed and validated using an existing quality database comprised of burn patients at risk for AKI. The database was derived from a hospital project to validate a NGAL biomarker assay for potential clinical laboratory implementation as a laboratory developed test. Patient population and methods of analysis are described below:

Study Population: The database consisted of 50 adult (age ≥18 years) patients with ≥20% total body surface area (TBSA) burns at risk for AKI. Plasma samples obtained as part of routine clinical basic metabolic panels were collected on the first hospital day and banked for additional testing. The focus on the first 24 hours was based on burn patient AKI risk immediately following injury to guide resuscitative measures, and to standardize creatinine testing results for comparison. Specifically, plasma creatinine testing was performed via the clinical laboratory using a Jaffe-based method (Beckman Coulter, Brea, Calif.) at admission. Serial creatinine testing on subsequent days were based on enzymatic method. Neutrophil gelatinase associated lipocalin (NGAL) concentrations were quantified using a commercially available enzyme-linked immunosorbant assay (Bioporto, Inc, Denmark). These NGAL results were not used for patient care. In brief, NGAL is released by neutrophils during inflammation and renally cleared.^{10, 11} Neutrophil gelatinase associated lipocalin clearance is reduced during AKI due to decreased GFR. Uniquely, renal tubular cells also produce NGAL during AKI—increasing both plasma and urine concentrations of NGAL through reabsorption and elimination respectively.

Given its role in cardiorenal syndrome, N-terminal pro B-type natriuretic peptide (NT-proBNP) was also measured (Roche Diagnostics, Indianapolis, Ind.) in the same plasma samples to complement NGAL. As with NGAL, NT-proBNP results were also not reported to the healthcare providers. Paired serial UOP measurements and vital signs were also collected from the electronic medical record (EMR). Chart review was used to determine which patients experienced AKI within a one-week period following burn intensive care unit admission. Acute kidney injury was defined using the Kidney Disease: Improving Global Outcomes (KDIGO) criteria as shown in Table 3:

TABLE 3
KDIGO Criteria

| Stage | Serum Creatinine | Urine Output |
|---|---|---|
| 1 | 1.5-1.9x baseline, or ≥0.3 mg/dL increase | <0.5 mL/kg/h for 6-12 h |
| 2 | 2.0-2.9x baseline | <0.5 mL/kg/h for ³12 h |
| 3 | 3.0x baseline, or an increase ≥4.0 mg/dL, initiation | <0.3 mL/kg/h for ³24 h, or |
| | of renal replacement therapy, in patients <18 years, | anuria for ³12 h |
| | decrease in eGFR to <35 mL/min per 1.73 m² | |

Abbreviations: eGFR, estimated glomerular filtration rate

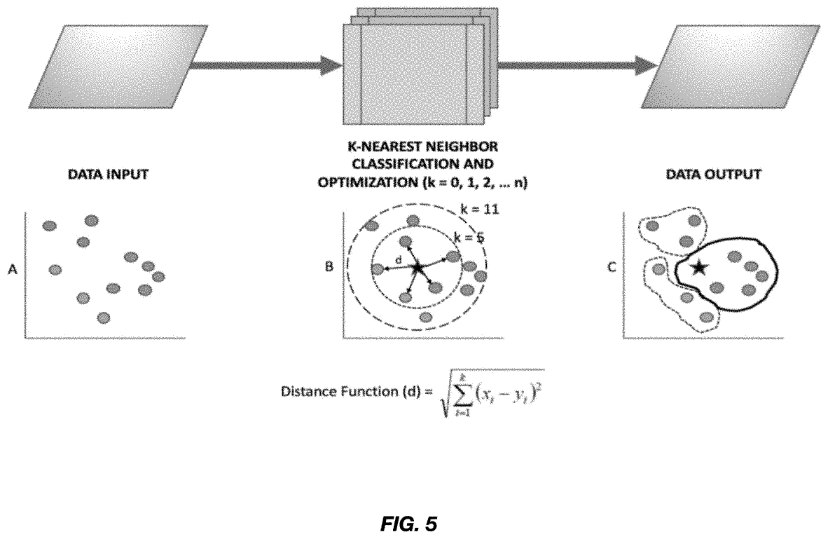

ML Algorithm: The Scikit-Learn's version 0.20.2 k-nearest neighbor (k-NN) algorithm was employed to build multiple ML models to classify and distinguish AKI from non-AKI cases, as illustrated in the block diagram of FIG. 5. Briefly, k-NN is a non-parametric pattern recognition algorithm used for classification and regression. For this example proof of concept, k-NN classified patients as having AKI or no AKI. Input for the algorithm consisted of k closest training examples from the same dataset, where k was the value equal to the square root of the number of instances as shown in FIG. 5. Once the data has been acquired, the training and testing steps in the k-INN algorithm involves the following steps:

1) Data points are normalized so that the distribution will ultimately have a mean value of 0 with a standard deviation of 1. This may be achieved in some implementations by subtracting the sample mean from each patient value and dividing by the standard deviation of the dataset. These data are then stored and split into training and testing sets (e.g., 80% for the training phase and 20% for the testing-validation accuracy phase or 60% for the training phase and 40% for the testing-validation accuracy phase;

2) Distance from a new data point (black “star” in FIG. 5) is then calculated against the stored data points in the training set;

3) the data points are then sorted based on an increasing order of distance from the new data point;

4) the majority of closest points distances (“k”: number of calculated closest points) assigns the new data point to the appropriate class. A Euclidian-based distance function (d) is applied to calculate the closest distance;

5) the final model is then tested against the unknown (20% or 40%) test sets to calculate the validation accuracy. The choice of k will affect the class assignment/validation accuracy;

6) a “k” optimizer is used to find the optimal “k” value that generated the most accurate model.

Hence, based on the above approach, patients were then classified by a majority vote of its neighbors, with subjects then being assigned to the class most common among these nearest neighbors (k). A defined subset of neighbors was then selected from the dataset for having AKI or no AKI. The algorithm was also applied with and without NGAL, NT-proBNP, creatinine, or UOP to determine which biomarker provided the best predictive classification ML model across a range of k-values. The validation accuracy of these ML models was then assessed on an unknown set of random test cases from the original study material that were not included in the training phase of the build. To further assess each feature's independent contribution to the ML model, as noted above, feature variations and training-testing set variations (80%−20% versus 60%−40%) were used to build multiple unique ML models (e.g., models built with just two features such as NT-proBNP and NGAL or those built with three features such as NGAL, NT-proBNP and creatinine, with varying number of k values etc.). This approach allowed building of 330 unique ML models (each with 22 feature and model selection variations×15 distinct k values) which were then compared and contrasted to each other to assess the significance of the individual features noted above and their significance in classifying new AKI cases. Each ML model was initially assessed through its training set accuracy and then subsequently tested against the unknown test set (the 20% or 40% unknown cases as mentioned in train-test split method noted above) to assess its validation accuracy.

Cross Validation studies: In addition to the aforementioned tests and validation studies, the individual categories (e.g., those with all 4 features versus those with all combination of 3 or 2 features) were also cross validated using the Scikit-learn cross validation grid search tool, enabling building and comparison of 10 unique models within each k value in each category to yield a total of 2,200 ML models. The mean accuracy for each set of these 10 models for a given k value in a given category was then analyzed.

Statistical Analysis: Statistical analysis was performed using IMP software (SAS Institute, Cary, N.C.). Descriptive statistics compared demographics between AKI versus non-AKI groups. Continuous variables were analyzed using the 2-sample t-test, while discrete variables were compared using the Chi-square test. Multivariate logistic regression was used to determine predictors of AKI with age and burn size serving as covariates. Repeated measures analysis of variance was used for time series data. A p-value <0.05 was considered statistically significant. Receiver operator characteristic (ROC) analysis was also performed to compare AKI biomarker performance.

Fifty percent of patients (25/50) in the dataset experienced AKI within the first week of hospital stay based on KDIGO criteria. Patient demographics are summarized in table 4:

TABLE 4
Patient Demographics

| | | | |
|---|---|---|---|
| Mean | | | |
| (SD) Variable | AKI Group | Non-AKI Group | P-Value |

| | | | | | |
|---|---|---|---|---|---|
| Age (years) | 39.1 | (49.2) | 39.7 | (15.5) | 0.922 |
| Burn Size (% TBSA) | 49.2 | (24.1) | 43.3 | (18.9) | 0.473 |

| | | | |
|---|---|---|---|
| Gender (M/F) | 20/5 | 19/6 | 0.832 |

| | | | | | |
|---|---|---|---|---|---|
| Mean Arterial | 78.9 | (11.5) | 80.1 | (5.2) | 0.782 |
| Pressure (mmHg) | | | | | |
| Central Venous | 13.3 | (3.4) | 12.0 | (7.6) | 0.662 |
| Pressure (mmHg) | | | | | |
| Creatinine | 1.21 | (0.51) | 0.90 | (0.22) | 0.066 |
| (mg/dL) | | | | | |
| NGAL | 185.1 | (86.3) | 110.3 | (48.1) | 0.013 |
| (ng/mL) | | | | | |
| NT-proBNP | 25.7 | (15.4) | 16.0 | (15.3) | 0.112 |
| (pg/mL) | | | | | |
| Urine Output | 81.5 | (31.6) | 85.7 | (48.9) | 0.795 |
| (mL/hr) | | | | | |

| | | | | |
|---|---|---|---|---|
| Time to AKI | 42.7 | (23.2) | N/A | N/A |
| (time from admission to | | | | |
| achieving AKI based | | | | |
| on KDIGO criteria in hours) | | | | |

Abbreviations: F, female; KDIGO, Kidney Disease: Improving Global Outcomes; M, male; NGAL, neutrophil gelatinase associated lipocalin; NA, not applicable; NT-proBNP; N-terminal pro-B-type natriuretic peptide; TBSA, total body surface area; RRT, renal replacement therapy.

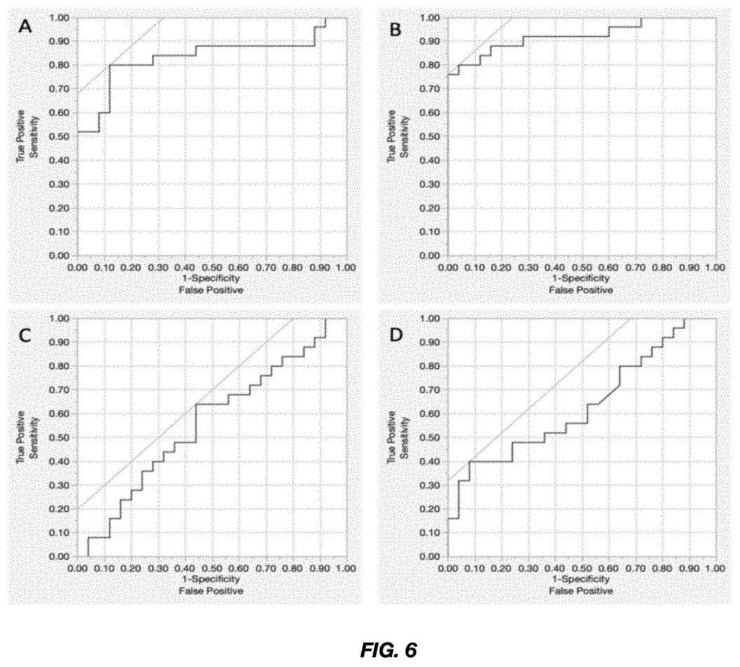

Plasma creatinine (1.21 [0.52] vs. 0.90 [0.22] mg/dL, P=0.066) and UOP (81.5 [31.6] vs. 85.7 [48.9] mL/hr, P=0.795) were not significantly different for samples obtained during the first day of admission for AKI versus non-AKI patients respectively. However, plasma creatinine (1.52 [0.66] vs. 0.83 [0.15] mg/dL, P=0.032) was significantly higher by day two for AKI patients. Based on plasma creatinine and/or UOP values obtained from the EMR, the average time for in the AKI group to achieve at least stage 1 KDIGO criteria was 42.7 (15.8) hours following burn intensive care unit admission. Multivariate logistic regression showed NGAL alone (OR 4.3, 95% CI 1.2-7.5, P=0.011) to be an independent predictor of AKI when adjusted for age and burn size. FIG. 6 illustrates graphs comparing ROC curves and AUC for BNP (Panel A), NGAL (Panel B), UOP (Panel C), and creatinine (Panel D). The area under the ROC curve showed NGAL providing significantly greater sensitivity and specificity (area under the curve: 0.92) compared to other biomarkers (BNP: 0.83, UOP: 0.56, and creatinine: 0.64).

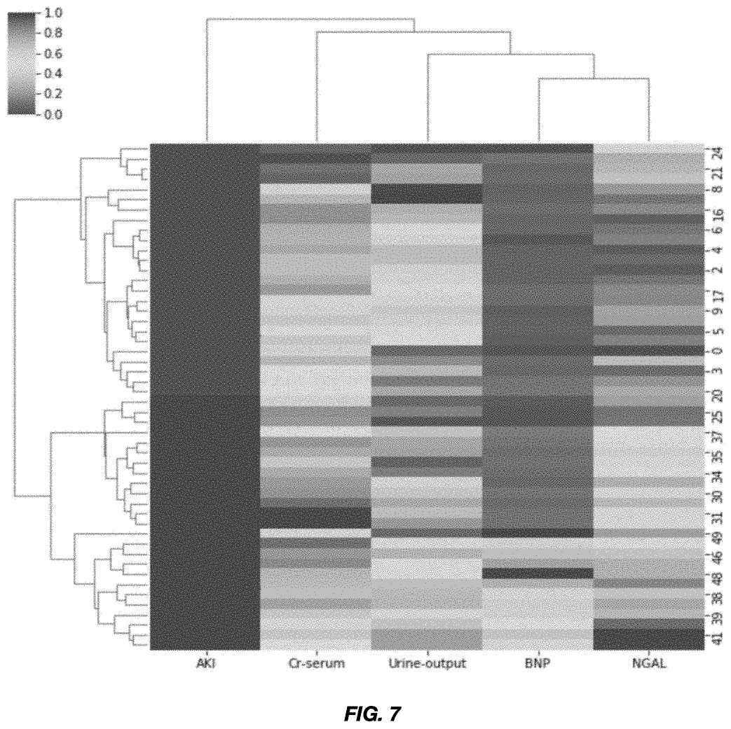

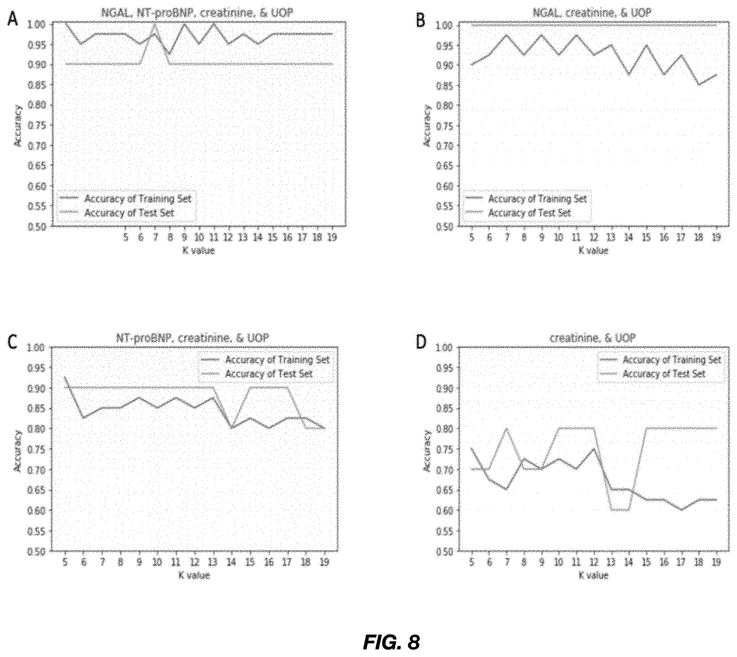

The correlation of the features and their relationship to AKI or No-AKI used in building the k-NN ML models is illustrated in the heat map shown in FIG. 7. FIG. 8 illustrates accuracy of this model for each of various biomarkers. Using the 80%−20% train-test split of results, the k-NN algorithm was found to maintain 90% accuracy when including NGAL, creatinine, UOP, and NT-proBNP for k-values ranging from 1 to 6, and 8 to 20 (Panel A). When k=7, the accuracy was 100% using the 80%−20% train-test set. With the same train-test split, the k-NN algorithm was found to consistently maintain 100% accuracy when excluding NT-proBNP (Panel B). Cross-validation studies also supported these findings with an average accuracy of 98% (5.4%) when all biomarkers were included. When using only NT-proBNP, UOP and creatinine, accuracy was 80 to 90% (Panel C), which was further supported by cross-validation study results showing an average accuracy of 88% (14.3%). The UOP and creatinine alone exhibited lowest accuracy ranging from 60 to 80% (Panel D) with a cross validation study results showing an average accuracy of 68% (19.4%).

Similar results were observed using a 60%-40% training-testing split, which showed accuracy ranging from 95 to 100% for k-values of 1 to 13 neighbors when NGAL, creatinine, NT-proBNP, and UOP were included. Without NGAL, accuracy decreased from 95 to 60% for k>13. Removing NT-proBNP from the algorithm decreased accuracy from 100 to 70% for k values >17. Accuracy varied from 80 to 100% when creatinine was removed from the algorithm across the range of k-values. The removal of UOP had the least impact on the ML algorithm with accuracy was maintained at 100% until k=15. When k>15, error rates of 10 to 35% were observed. Similar to the 80%−20% train-test set analysis of biomarker pairs, we see algorithms using the 60%−40% achieving an accuracy ranging from 90 to 100% when NGAL was included. When creatinine and NT-proBNP were used together and excluding NGAL and UOP, an accuracy ranging from 85 to 90% was achieved for k-values ranging from 5 to 13.

The average accuracies obtained from cross-validation studies for the 2,200 ML models for the categories noted above further verified the above trends and results.

Accordingly, implementations of AI/ML models may provide advantages for predicting burn related AKI. Machine learning in particular offers several advantages over human-based decision making. Advantages include high automation and early classification of subtle changes or patterns via computer-based AKI recognition. Evaluating the burn AKI dataset discussed above using a k-NN ML algorithm provides a pragmatic and innovative approach that analyzes traditional indicators of renal dysfunction (e.g., creatinine and UOP) as well as novel biomarkers of kidney injury (e.g., NGAL) targeting the critical first 24 hours following injury.

NGAL was shown to be a statistically useful biomarker for predicting AKI on the first day of burn intensive care unit (ICU) admission. Enhanced predictive performance of NGAL was also reflected with the k-NN ML algorithm with classification accuracy approaching 100% even without NT-proBNP and UOP.

Urine output has been known to be a poor predictor of AKI especially during acute burn resuscitation. Glomerular filtration rate may be altered despite UOP remaining normal due to neurohormonal autoregulation. Thus, in many implementations, the exclusion of UOP may enhance performance of the ML algorithm.

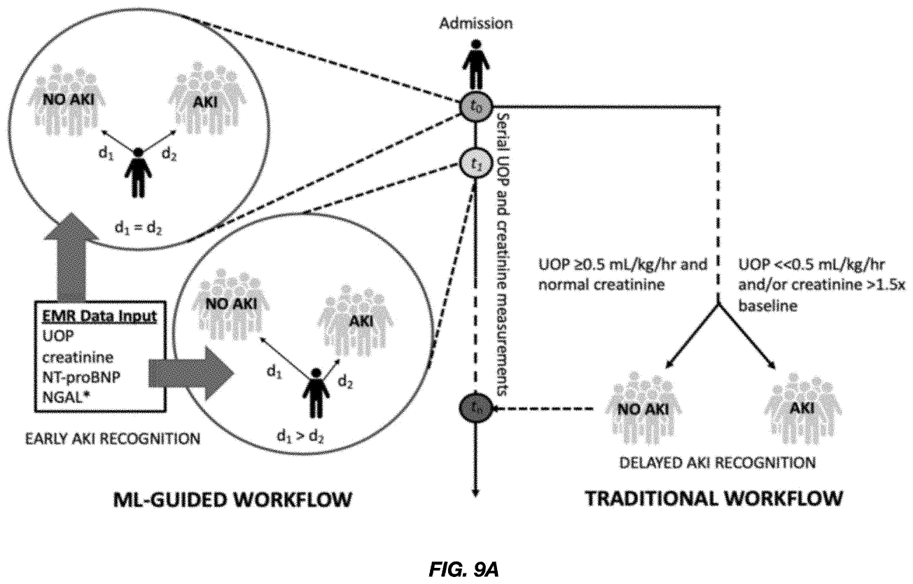

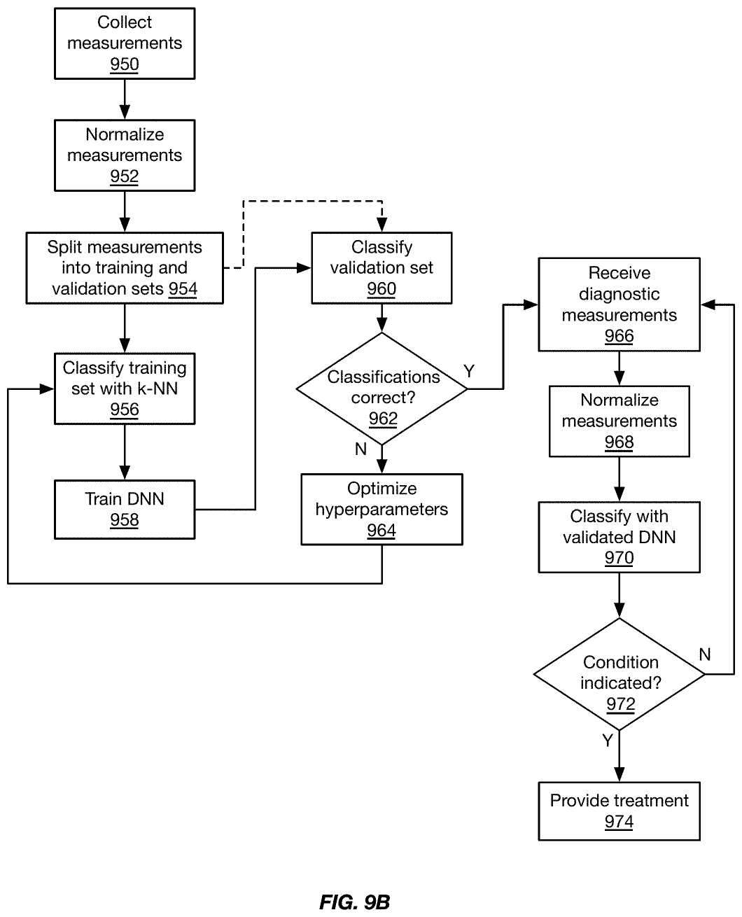

The ML algorithms discussed herein may also serve as a useful supplement for the NGAL biomarker especially in the pre-hospital setting where UOP, creatinine and NT-proBNP may be performed at the point of care. FIG. 9A illustrates the conceptual role of an ML algorithm for burn AKI recognition. Careful selection identification of acceptable k neighbors (based on the k optimizer approach) along with cross validation study may allow avoiding values that could adversely affect the model's accuracy. The feature variations and the two distinct train-test split platforms (80%−20% and 60%−40%) along with the cross-validation studies for the range of k values, allowed us build and evaluate over 2,200 unique ML models. Both training-testing sets of 60%−40% and 80%−20% provided acceptable balance for classifying burn AKI. The overall trend noted in 60%−40% and 80%−20% train-test split models showed similar patterns with respect to the feature variations (e.g., enhanced accuracy with NGAL and reduced accuracy when using UOP as parameters) which were further supported by cross-validation results.

Using UOP along with plasma creatinine and NT-proBNP, models were constructed that were able to achieve up to 90% accuracy within the two train-test split categories as shown in FIG. 8, which could classify new patients as either AKI versus no-AKI within the first 24 hours—faster than the average 42.7 (23.2) hours required for these patients to meet KDIGO AKI criteria. With only NT-proBNP and creatinine, the ML algorithm achieved an accuracy ranging from 85 to 90% for samples obtained in the same 24-hour time period. Given the widespread availability of creatinine, UOP, and NT-proBNP measurements, ML could serve as a surrogate tool to enhance burn AKI recognition in routine clinical practice and in the absence of NGAL.

The advent of EMR serves as a double-edged sword. It may be difficult in some instances for physicians or users to integrate more than seven pieces of information at any given time. In part, EMR systems have provided means to capture and organize the substantial volumes of medical information. However, as the number of laboratory tests grow along with other health information, the EMR becomes overwhelming for providers and prevents conversion of these data into timely and clinically actionable knowledge. Artificial intelligence overcomes these human limitations. With advances in portable computing power, AI/ML may be employed as part of EMR decision support and/or handheld smart devices to augment decision making at the bedside. Accordingly, AI/ML has clinical utility for burn-related AKI when using just a few routine laboratory results.

FIG. 9B is a flow chart of an implementation of a method for machine-learning based diagnosis and treatment. At step 950, measurements of biomarkers and vital signs from a population with a known clinical diagnosis may be obtained. The biomarkers and vital signs may include any type and form of biomarkers, including NGAL, UOP, creatinine, NT-proBNP, temperature, heart rate, O2 saturation, or any other type and form of biomarker or vital sign or combinations of biomarkers or vital signs. The measurements may be obtained from a database on the same computing device or over a network, e.g. from a public database in some implementations. Data sources may be of any type and form, including clinical laboratory projects, patient histories, etc. Each measurement may be associated with a clinical diagnosis or indication of a condition (or absence of a condition).

At step 952, in some implementations, each measurement may be normalized. Normalization may comprise scaling measurements within a predetermined range, e.g. linearly or geometrically scaling or otherwise normalizing measurements, depending on implementation. In some implementations, measurements may be scaled by adjusting a mean measurement value to 0 with a predetermined standard deviation value (e.g. 1, such that one standard deviation is from −1 to 1). Normalization may also include other processing steps, such as filtering or excluding extreme values (e.g. those beyond two or three standard deviations from the mean) to reduce variability, in some implementations. Other ranges and mean values may also be utilized.

At step 954, the normalized measurements may be subdivided into two sets, one for training and one for validation. Each set may be approximately equal in size in some implementations, while in other implementations, either set may be larger. Each set may comprise a subset of population measurements and corresponding diagnoses or indications, and may be selected randomly in many implementations. In other implementations, some measurement sets may be incomplete, and more complete sets may be selected for the training set in some implementations. In some implementations, the training set may be from a subset of the population, such as patients that experienced or did not experience acute kidney injury following a significant burn injury, while the validation set may be from a larger or more generalized subset of the population (e.g. patients that experienced or did not experience acute kidney injury, regardless of initial trauma, including burns, surgical trauma, or other incidents). This may allow the machine learning system to be generalized to larger populations during the validation phase, as discussed above.

At step 956, features of the training set may be analyzed, e.g. via a k-NN pattern recognition algorithm. A point in a multi-dimensional space corresponding to a measurement set for an individual may be selected (e.g. with a number of dimensions corresponding to the number of biometric or vital sign types, in some implementations), and a distance calculated to each neighboring measurement point within the multi-dimensional space. In some implementations, the number of dimensions may be reduced to reduce the scale of the analysis (e.g. via principal component analysis, linear discriminant analysis, or canonical correlation analysis). The distances to each neighboring measurement point may be sorted in order, and the measurement point may be classified according to a majority of its k-nearest neighbors. The process may be iterated for each additional measurement point in some implementations. In some implementations, a decision boundary may be calculated for classification of additional or future measurement points.

At step 958, a deep neural network may be trained according to the results of the k-NN classification. The network may have any number of hidden layers, and may receive as inputs each of the normalized values for biomarkers or vital signs from the training set, and may provide a positive or negative value for the indication or diagnosis. The training may be performed recursively with internodal weights adjusted at each iteration to optimize the classification results (e.g. for accuracy or sensitivity, in various implementations) until a predetermined accuracy or sensitivity threshold is reached, or for a predetermined number of iterations.

At step 960, the second set of measurements (e.g. the validation set) may be classified according to the trained network, and the classification results compared to the known diagnosis or indication (or absence of an indication). A confusion matrix may be generated in some implementations, and an accuracy, sensitivity, precision, or similar metric may be compared to a predetermined threshold at step 962. If the metric does not exceed the threshold (e.g. if the classifications are not correct, for the desired metric and threshold), then the hyperparameters of the k-NN and/or DNN may be adjusted, and steps 956-962 may be repeated. If the metric exceeds the threshold, then the model may be validated and used for diagnostics.

At step 966, a set of measurements of biometrics or vital signs for an individual or population may be received with an unknown diagnosis for a condition or indication. At step 968, the measurements may be normalized or scaled, as discussed above at step 952, and at step 970, the set of measurements may be classified via the validated neural network. At step 972, the system may determine if the condition is indicated by the classification (e.g. if the classification value exceeds a threshold, in many implementations). If so, then at step 974, a treatment may be provided. For example, in implementations analyzing and diagnosing or predicting acute kidney injury, a treatment such as a course of increased fluid administration, plasmapheresis or plasma exchange may be provided. Via the machine-learning based analysis system, such treatments may be provided an earlier time or stage than in implementations in which physicians must weight for further measurements or verifications, which may drastically improve patient outcomes. Similarly, by accurately classifying individuals who do not have an indication, treatments that may be potentially harmful or risky for individuals lacking the indication may be avoided.

Accordingly, the systems and methods discussed herein may be used to predict or diagnose acute kidney injury from any source, including burns or trauma, providing physicians the opportunity to begin treatment at an early stage, long before a diagnosis would be available when not implementing these systems and methods. Additionally, although primarily discussed above in connection with acute kidney injury, the systems and methods discussed herein may be generalized or applied to any type and form of indication or diagnosis, including early identification of sepsis, myocardial infarctions, coagulopathy, or any other indication. In particular, in many implementations, the systems and methods discussed herein may be applied to early-stage diagnosis and treatment of acute kidney injury, including acute kidney injury based on or a result of pre-renal, intrinsic, and post-renal causes. In addition to burn and trauma patients, this may include kidney transplant patients, surgery patients, patients in intensive care units, oncology patients, cardiology patients, diabetic patients, patients with chronic kidney disease, or any other population or subgroup, including patients of any demographic or age including elderly patients and infants or newborn patients. Examples of pre-renal causes (e.g. those occurring upstream of the kidneys, or in the blood supply) may include decreased blood volume or hypovolemia, which may be a result of trauma, shock, dehydration and fluid loss, or excessive diuretics use; liver failure or hepatorenal syndrome impairing renal perfusion; atheroembolic disease or renal vein thrombosis or other vascular problems, including innate vascular problems or those occurring as a result of nephrotic syndrome; severe burns; sequestration as a result of pericarditis or pancreatitis or any other similar inflammation; and hypotension, which may occur as a result of antihypertensives or vasodilators. Examples of intrinsic causes or those causing damage directly to the kidneys include toxins or medications (e.g. nonsteroidal anti-inflammatory drugs, aminoglycoside antibiotics, iodinated contrast, lithium, phosphate nephropathy due to bowel preparation for colonoscopy with sodium phosphates, statins, stimulants, or other medications or toxins, including environmental toxins); breakdown of muscle tissue or rhabdomyolysis, with an increased release of myoglobin in the blood; traumatic injury, including crush injuries or blunt trauma; breakdown of red blood cells or hemolysis, as a result of sickle-cell disease, lupus erythematosus, or other conditions; multiple myeloma, for example due to hypercalcemia or cast nephropathy; and acute glomerulonephritis which may be due to a variety of causes, such as anti-glomerular basement membrane disease or Goodpasture's syndrome, Wegener's granulomatosis or acute lupus nephritis with systemic lupus erythematosus. Examples of post-renal causes or those affecting the urinary tract, including medication interfering with normal bladder emptying (e.g. anticholinergics); benign prostatic hypertrophy or prostate cancer; kidney stones; ovarian cancer, colorectal cancer, or other abdominal conditions; obstructed urinary catheters; and drugs that can cause crystalluria, myoglobinuria, cystitis, or other such conditions.

In one aspect, the present disclosure is directed to a method for training a neural network for early recognition of acute kidney injury comprising. The method includes collecting a set of biomarker and vital sign measurements of a population with a known clinical diagnosis. The method also includes applying one or more transformations to each biomarker and vital sign measurement including normalization to create a modified set of biomarker and vital sign measurements. The method also includes creating a first training set comprising a subset of the modified set of biomarker and vital sign measurements. The method also includes, for each of a plurality of measurements of the subset, calculating a distance from a selected measurement of the subset. The method also includes sorting each of the plurality of measurements of the subset based on an increasing order of distance from the selected measurement of the subset. The method also includes classifying a further subset of the subset based on the sorted distance as belonging to a first class. The method also includes training the neural network in a first stage using the first training set. The method also includes creating a second training set for a second stage of training comprising a second subset of the modified set of biomarker and vital sign measurements. The method also includes validating the neural network in a second stage using the second training set.

In some implementations, the set of biomarker and vital sign measurements comprise at least one of neutrophil gelatinase associated lipocalin (NGAL), urine output (UOP), creatinine, and N-terminal B-type natriuretic peptide (NT-proBNP). In some implementations, the method includes scaling each biomarker and vital sign measurement to a predetermined range. In a further implementation, the method includes, for each biomarker and vital sign measurement, dividing a difference between a mean value of the corresponding measurements and the measurement by a standard deviation of the corresponding measurements.

In some implementations, the method includes assigning the further subset to the first class based on a majority of a predetermined number of the sorted measurements being associated with the first class. In some implementations, the method includes classifying each of the second subset of the modified set of biomarker and vital sign measurements with the trained neural network, and determining whether the classifications correspond to the known clinical diagnoses.

In some implementations, at least one of the biomarker and vital sign measurements is not independently correlated with the known clinical diagnoses. In some implementations, the method includes receiving biomarker and vital sign measurements of an individual with an unknown clinical diagnosis; and classifying the individual with the biomarker and vital sign measurements according to the validated neural network. In a further implementation, at least one treatment is provided responsive to the classification corresponding to acute kidney injury. In a still further implementation at least one treatment comprises a course of increased fluid administration, plasmapheresis, or plasma exchange. Alternately, increasing intravenous fluids may also help mitigate AKI in cases of severe burns and trauma.

In another aspect, the present disclosure is directed to a system for training a neural network for early recognition of acute kidney injury. The system includes a computing device comprising a processor and a memory device storing a set of biomarker and vital sign measurements of a population with a known clinical diagnosis. The processor is configured to: apply one or more transformations to each biomarker and vital sign measurement including normalization to create a modified set of biomarker and vital sign measurements; create a first training set comprising a subset of the modified set of biomarker and vital sign measurements; for each of a plurality of measurements of the subset, calculate a distance from a selected measurement of the subset; sort each of the plurality of measurements of the subset based on an increasing order of distance from the selected measurement of the subset; classify a further subset of the subset based on the sorted distance as belonging to a first class; train the neural network in a first stage using the first training set; create a second training set for a second stage of training comprising a second subset of the modified set of biomarker and vital sign measurements; and validate the neural network in a second stage using the second training set.

In some implementations, the set of biomarker and vital sign measurements comprise at least one of neutrophil gelatinase associated lipocalin (NGAL), urine output (UOP), creatinine, and N-terminal B-type natriuretic peptide (NT-proBNP). In some implementations, the processor is further configured to scale each biomarker and vital sign measurement to a predetermined range. In a further implementation, the processor is further configured to scale each biomarker and vital sign measurement to a predetermined range by, for each biomarker and vital sign measurement, dividing a difference between a mean value of the corresponding measurements and the measurement by a standard deviation of the corresponding measurements.

In some implementations, the processor is further configured to assign the further subset to the first class based on a distance between each measurement of the further subset being less than a threshold. In some implementations, the processor is further configured to validate the neural network in the second stage by classifying each of the second subset of the modified set of biomarker and vital sign measurements with the trained neural network, and determining whether the classifications correspond to the known clinical diagnoses. In some implementations, at least one of the biomarker and vital sign measurements is not independently correlated with the known clinical diagnoses. In some implementations, the processor is further configured to: receive biomarker and vital sign measurements of an individual with an unknown clinical diagnosis, and classify the individual with the biomarker and vital sign measurements according to the validated neural network; and at least one treatment is provided, responsive to the classification corresponding to acute kidney injury.

In still another aspect, the present disclosure is directed to a method for early treatment of acute kidney injury. The method includes receiving biomarker and vital sign measurements of an individual with an unknown clinical diagnosis; and classifying the individual as corresponding to acute kidney injury via a trained neural network, wherein the neural network is trained in a first stage using a first training set comprising a first subset of a set of biomarker and vital sign measurements of a population with a known clinical diagnosis, and validated in a second stage using a second training set comprising a second subset of the set of biomarker and vital sign measurements of the population with the known clinical diagnosis, wherein each biomarker and vital sign measurement is normalization to create a modified set of biomarker and vital sign measurements prior to the first stage and second stage. At least one treatment is provided responsive to the classification corresponding to acute kidney injury. In some implementations, the at least one treatment comprises a course of increased fluid administration, plasmapheresis, or plasma exchange.

### C. Computing Environment

Having discussed specific embodiments of the present solution, it may be helpful to describe aspects of the operating environment as well as associated system components (e.g., hardware elements) in connection with the methods and systems described herein.

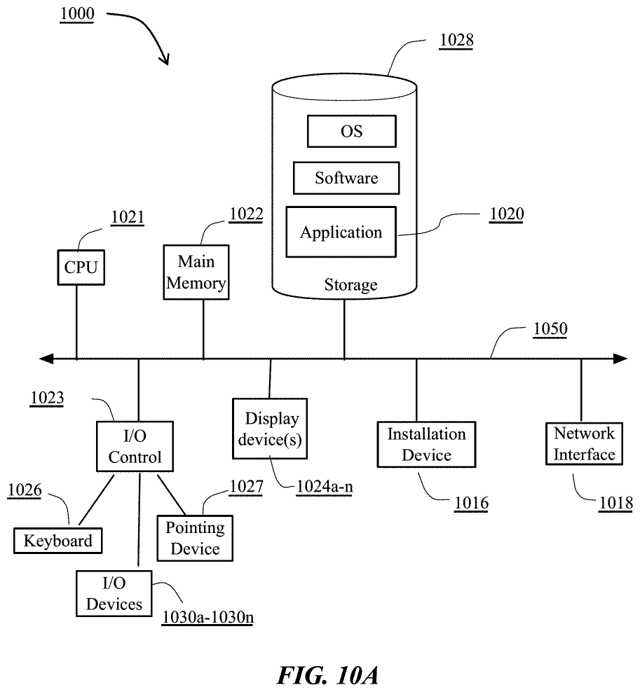

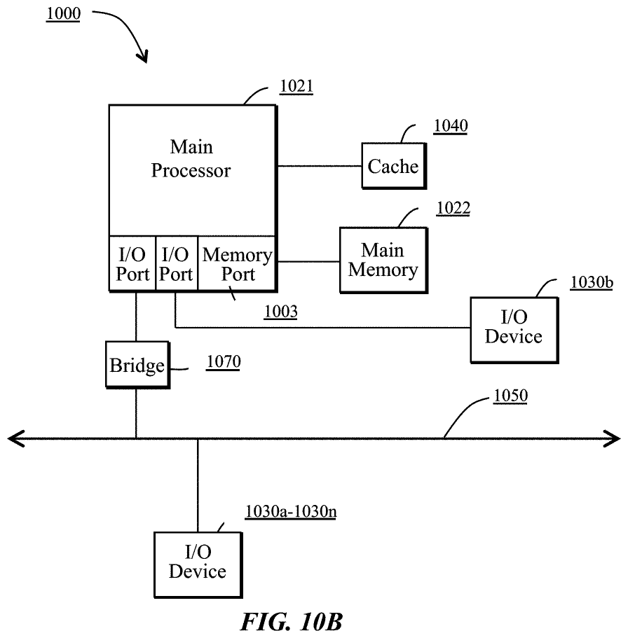

The systems discussed herein may be deployed as and/or executed on any type and form of computing device, such as a computer, network device or appliance capable of communicating on any type and form of network and performing the operations described herein. FIGS. 10A and 10B depict block diagrams of a computing device 1000 useful for practicing an embodiment of the wireless communication devices 1002 or the access point 1006. As shown in FIGS. 10A and 10B, each computing device 1000 includes a central processing unit 1021, and a main memory unit 1022. As shown in FIG. 10A, a computing device 1000 may include a storage device 1028, an installation device 1016, a network interface 1018, an I/O controller 1023, display devices 1024a-1024n, a keyboard 1026 and a pointing device 1027, such as a mouse. The storage device 1028 may include, without limitation, an operating system and/or software. As shown in FIG. 10B, each computing device 1000 may also include additional optional elements, such as a memory port 1003, a bridge 1070, one or more input/output devices 1030a-1030n (generally referred to using reference numeral 1030), and a cache memory 1040 in communication with the central processing unit 1021.

The central processing unit 1021 is any logic circuitry that responds to and processes instructions fetched from the main memory unit 1022. In many embodiments, the central processing unit 1021 is provided by a microprocessor unit, such as: those manufactured by Intel Corporation of Mountain View, Calif.; those manufactured by International Business Machines of White Plains, N.Y.; or those manufactured by Advanced Micro Devices of Sunnyvale, Calif. The computing device 1000 may be based on any of these processors, or any other processor capable of operating as described herein.

Main memory unit 1022 may be one or more memory chips capable of storing data and allowing any storage location to be directly accessed by the microprocessor 1021, such as any type or variant of Static random access memory (SRAM), Dynamic random access memory (DRAM), Ferroelectric RAM (FRAM), NAND Flash, NOR Flash and Solid State Drives (SSD). The main memory 1022 may be based on any of the above described memory chips, or any other available memory chips capable of operating as described herein. In the embodiment shown in FIG. 10A, the processor 1021 communicates with main memory 1022 via a system bus 1050 (described in more detail below). FIG. 10B depicts an embodiment of a computing device 1000 in which the processor communicates directly with main memory 1022 via a memory port 1003. For example, in FIG. 10B the main memory 1022 may be DRDRAM.

FIG. 10B depicts an embodiment in which the main processor 1021 communicates directly with cache memory 1040 via a secondary bus, sometimes referred to as a backside bus. In other embodiments, the main processor 1021 communicates with cache memory 1040 using the system bus 1050. Cache memory 1040 typically has a faster response time than main memory 1022 and is provided by, for example, SRAM, B SRAM, or EDRAM. In the embodiment shown in FIG. 10B, the processor 1021 communicates with various I/O devices 1030 via a local system bus 1050. Various buses may be used to connect the central processing unit 1021 to any of the I/O devices 1030, for example, a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 1024, the processor 1021 may use an Advanced Graphics Port (AGP) to communicate with the display 1024. FIG. 10B depicts an embodiment of a computer 1000 in which the main processor 1021 may communicate directly with I/O device 1030b, for example via HYPERTRANSPORT, RAPIDIO, or INFINIBAND communications technology. FIG. 10B also depicts an embodiment in which local busses and direct communication are mixed: the processor 1021 communicates with I/O device 1030a using a local interconnect bus while communicating with I/O device 1030b directly.

A wide variety of I/O devices 1030a-1030n may be present in the computing device 1000. Input devices include keyboards, mice, trackpads, trackballs, microphones, dials, touch pads, touch screen, and drawing tablets. Output devices include video displays, speakers, inkjet printers, laser printers, projectors and dye-sublimation printers. The I/O devices may be controlled by an I/O controller 1023 as shown in FIG. 10A. The I/O controller may control one or more I/O devices such as a keyboard 1026 and a pointing device 1027, e.g., a mouse or optical pen. Furthermore, an I/O device may also provide storage and/or an installation medium 1016 for the computing device 1000. In still other embodiments, the computing device 1000 may provide USB connections (not shown) to receive handheld USB storage devices such as the USB Flash Drive line of devices manufactured by Twintech Industry, Inc. of Los Alamitos, Calif.

Referring again to FIG. 10A, the computing device 1000 may support any suitable installation device 1016, such as a disk drive, a CD-ROM drive, a CD-R/RW drive, a DVD-ROM drive, a flash memory drive, tape drives of various formats, USB device, hard-drive, a network interface, or any other device suitable for installing software and programs. The computing device 1000 may further include a storage device, such as one or more hard disk drives or redundant arrays of independent disks, for storing an operating system and other related software, and for storing application software programs such as any program or software 1020 for implementing (e.g., configured and/or designed for) the systems and methods described herein. Optionally, any of the installation devices 1016 could also be used as the storage device. Additionally, the operating system and the software can be run from a bootable medium.

Furthermore, the computing device 1000 may include a network interface 1018 to interface to the network 1004 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, IEEE 802.11ac, IEEE 802.11ad, CDMA, GSM, WiMax and direct asynchronous connections). In one embodiment, the computing device 1000 communicates with other computing devices 1000′ via any type and/or form of gateway or tunneling protocol such as Secure Socket Layer (SSL) or Transport Layer Security (TLS). The network interface 1018 may include a built-in network adapter, network interface card, PCMCIA network card, card bus network adapter, wireless network adapter, USB network adapter, modem or any other device suitable for interfacing the computing device 1000 to any type of network capable of communication and performing the operations described herein.

In some embodiments, the computing device 1000 may include or be connected to one or more display devices 1024a-1024n. As such, any of the I/O devices 1030a-1030n and/or the I/O controller 1023 may include any type and/or form of suitable hardware, software, or combination of hardware and software to support, enable or provide for the connection and use of the display device(s) 1024a-1024n by the computing device 1000. For example, the computing device 1000 may include any type and/or form of video adapter, video card, driver, and/or library to interface, communicate, connect or otherwise use the display device(s) 1024a-1024n. In one embodiment, a video adapter may include multiple connectors to interface to the display device(s) 1024a-1024n. In other embodiments, the computing device 1000 may include multiple video adapters, with each video adapter connected to the display device(s) 1024a-1024n. In some embodiments, any portion of the operating system of the computing device 1000 may be configured for using multiple displays 1024a-1024n. One ordinarily skilled in the art will recognize and appreciate the various ways and embodiments that a computing device 1000 may be configured to have one or more display devices 1024a-1024n.

In further embodiments, an I/O device 1030 may be a bridge between the system bus 1050 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, a USB connection, or a HDMI bus.

A computing device 1000 of the sort depicted in FIGS. 10A and 10B may operate under the control of an operating system, which control scheduling of tasks and access to system resources. The computing device 1000 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, produced by Google Inc.; WINDOWS 7 and 8, produced by Microsoft Corporation of Redmond, Wash.; MAC OS, produced by Apple Computer of Cupertino, Calif.; WebOS, produced by Research In Motion (RIM); OS/2, produced by International Business Machines of Armonk, N.Y.; and Linux, a freely-available operating system distributed by Caldera Corp. of Salt Lake City, Utah, or any type and/or form of a Unix operating system, among others.

The computer system 1000 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 1000 has sufficient processor power and memory capacity to perform the operations described herein.

In some embodiments, the computing device 1000 may have different processors, operating systems, and input devices consistent with the device. For example, in one embodiment, the computing device 1000 is a smart phone, mobile device, tablet or personal digital assistant. In still other embodiments, the computing device 1000 is an Android-based mobile device, an iPhone smart phone manufactured by Apple Computer of Cupertino, Calif., or a Blackberry or WebOS-based handheld device or smart phone, such as the devices manufactured by Research In Motion Limited. Moreover, the computing device 1000 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

Although the disclosure may reference one or more “users”, such “users” may refer to user-associated devices, for example, consistent with the terms “user” and “multi-user” typically used in the context of a multi-user multiple-input and multiple-output (MU-MIMO) environment.

It should be noted that certain passages of this disclosure may reference terms such as “first” and “second” in connection with devices, mode of operation, transmit chains, antennas, etc., for purposes of identifying or differentiating one from another or from others. These terms are not intended to merely relate entities (e.g., a first device and a second device) temporally or according to a sequence, although in some cases, these entities may include such a relationship. Nor do these terms limit the number of possible entities (e.g., devices) that may operate within a system or environment.

It should be understood that the systems described above may provide multiple ones of any or each of those components and these components may be provided on either a standalone machine or, in some embodiments, on multiple machines in a distributed system. In addition, the systems and methods described above may be provided as one or more computer-readable programs or executable instructions embodied on or in one or more articles of manufacture. The article of manufacture may be a floppy disk, a hard disk, a CD-ROM, a flash memory card, a PROM, a RAM, a ROM, or a magnetic tape. In general, the computer-readable programs may be implemented in any programming language, such as LISP, PERL, C, C++, C #, PROLOG, or in any byte code language such as JAVA. The software programs or executable instructions may be stored on or in one or more articles of manufacture as object code.

While the foregoing written description of the methods and systems enables one of ordinary skill to make and use what is considered presently to be the best mode thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

We claim:

1. A method for training a neural network for early recognition of acute kidney injury comprising:
collecting a set of biomarker and vital sign measurements of a population with a known clinical diagnosis;
applying one or more transformations to each biomarker and vital sign measurement including normalization to create a modified set of biomarker and vital sign measurements;
creating a first training set comprising a subset of the modified set of biomarker and vital sign measurements;
for each of a plurality of measurements of the subset, calculating a distance from a selected measurement of the subset;
sorting each of the plurality of measurements of the subset based on an increasing order of distance from the selected measurement of the subset;
classifying a further subset of the subset based on the sorted distance as belonging to a first class;
training the neural network in a first stage using the first training set;
creating a second training set for a second stage of training comprising a second subset of the modified set of biomarker and vital sign measurements; and
validating the neural network in a second stage using the second training set.

2. The method of claim 1, wherein the set of biomarker and vital sign measurements comprise at least one of neutrophil gelatinase associated lipocalin (NGAL), urine output (UOP), creatinine, and N-terminal B-type natriuretic peptide (NT-proBNP).

3. The method of claim 1, wherein applying the one or more transformations to each biomarker and vital sign measurements comprises scaling each biomarker and vital sign measurement to a predetermined range.

4. The method of claim 3, wherein scaling each biomarker and vital sign measurement to a predetermined range further comprises, for each biomarker and vital sign measurement, dividing a difference between a mean value of the corresponding measurements and the measurement by a standard deviation of the corresponding measurements.

5. The method of claim 1, wherein classifying the further subset further comprises assigning the further subset to the first class based on a majority of a predetermined number of the sorted measurements being associated with the first class.

6. The method of claim 1, wherein validating the neural network in the second stage comprises classifying each of the second subset of the modified set of biomarker and vital sign measurements with the trained neural network, and determining whether the classifications correspond to the known clinical diagnoses.

7. The method of claim 1, wherein at least one of the biomarker and vital sign measurements is not independently correlated with the known clinical diagnoses.

8. The method of claim 1, further comprising:
receiving biomarker and vital sign measurements of an individual with an unknown clinical diagnosis; and
classifying the individual with the biomarker and vital sign measurements according to the validated neural network.

9. The method of claim 8, wherein at least one treatment is provided responsive to the classification corresponding to acute kidney injury.

10. The method of claim 9, wherein the at least one treatment comprises a course of increased fluid administration, plasmapheresis, or plasma exchange.

11. A system for training a neural network for early recognition of acute kidney injury comprising:
a computing device comprising a processor and a memory device storing a set of biomarker and vital sign measurements of a population with a known clinical diagnosis;
wherein the processor is configured to:
apply one or more transformations to each biomarker and vital sign measurement including normalization to create a modified set of biomarker and vital sign measurements,
create a first training set comprising a subset of the modified set of biomarker and vital sign measurements,
for each of a plurality of measurements of the subset, calculate a distance from a selected measurement of the subset,
sort each of the plurality of measurements of the subset based on an increasing order of distance from the selected measurement of the subset,
classify a further subset of the subset based on the sorted distance as belonging to a first class,
train the neural network in a first stage using the first training set,
create a second training set for a second stage of training comprising a second subset of the modified set of biomarker and vital sign measurements, and
validate the neural network in a second stage using the second training set.

12. The system of claim 11, wherein the set of biomarker and vital sign measurements comprise at least one of neutrophil gelatinase associated lipocalin (NGAL), urine output (UOP), creatinine, and N-terminal B-type natriuretic peptide (NT-proBNP).

13. The system of claim 11, wherein the processor is further configured to scale each biomarker and vital sign measurement to a predetermined range.

14. The system of claim 13, wherein the processor is further configured to scale each biomarker and vital sign measurement to a predetermined range by, for each biomarker and vital sign measurement, dividing a difference between a mean value of the corresponding measurements and the measurement by a standard deviation of the corresponding measurements.

15. The system of claim 11, wherein the processor is further configured to assign the further subset to the first class based on a majority of a predetermined number of the sorted measurements being associated with the first class.

16. The system of claim 11, wherein the processor is further configured to validate the neural network in the second stage by classifying each of the second subset of the modified set of biomarker and vital sign measurements with the trained neural network, and determining whether the classifications correspond to the known clinical diagnoses.

17. The system of claim 11, wherein at least one of the biomarker and vital sign measurements is not independently correlated with the known clinical diagnoses.

18. The system of claim 11, wherein the processor is further configured to:
receive biomarker and vital sign measurements of an individual with an unknown clinical diagnosis, and
classify the individual with the biomarker and vital sign measurements according to the validated neural network; and
wherein at least one treatment is provided, responsive to the classification corresponding to acute kidney injury.

19. A method for early treatment of acute kidney injury, comprising:
receiving biomarker and vital sign measurements of an individual with an unknown clinical diagnosis; and
classifying the individual as corresponding to acute kidney injury via a trained neural network, wherein the neural network is trained in a first stage using a first training set comprising a first subset of a set of biomarker and vital sign measurements of a population with a known clinical diagnosis, and validated in a second stage using a second training set comprising a second subset of the set of biomarker and vital sign measurements of the population with the known clinical diagnosis, wherein each biomarker and vital sign measurement is normalization to create a modified set of biomarker and vital sign measurements prior to the first stage and second stage; and
wherein at least one treatment is provided responsive to the classification corresponding to acute kidney injury.

20. The method of claim 19, wherein the at least one treatment comprises a course of increased fluid administration, plasmapheresis, or plasma exchange.
